(19) United States
(12) Patent Application Publication
(10) Pub. No.: US 2008/0021455 A1
(43) Pub. Date: Jan. 24, 2008

(54) Articulating Sacral or Iliac Connector

(75) Inventors: Nam T. Chao, Marlborough, MA (US); Simon Siu, Quincy, MA (US); Praveen Mummaneni, Atlanta, GA (US); Ross Sylvia, Taunton, MA (US); Nicholas Pavento, Walpole, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DEPUY SPINE, INC., Raynham, MA (US)

(21) Appl. No.: 11/459,174

(22) Filed: Jul. 21, 2006

(51) Int. Cl. A61F 2/30

(52) U.S. Cl. 606/61

(57) ABSTRACT

Various methods and devices are provided for facilitating movement of a spinal connector to allow for coupling of the connector to spinal fixation elements and anchors. In one exemplary embodiment, a spinal connector is provided having a receiver head with opposed sidewalls defining a seating portion configured to seat a spinal fixation element. The spinal connector also includes a connecting rod extending from one of the sidewalls of the receiver head and having a first end coupled to the receiver head and a second end configured to couple to a spinal anchor.

### FIELD OF THE INVENTION

The present invention relates to spinal connectors for mating a spinal fixation element to bone.

### BACKGROUND

Spinal deformities, which include rotation, angulation, and/or curvature of the spine, can result from various disorders, including, for example, scoliosis (abnormal curvature in the coronal plane of the spine), kyphosis (backward curvature of the spine), and spondylolisthesis (forward displacement of a lumbar vertebra). Other causes of an abnormally shaped spine include trauma and spinal degeneration with advancing age. Early techniques for correcting such deformities utilized external devices that applied force to the spine in an attempt to reposition the vertebrae. These devices, however, resulted in severe restriction and in some cases immobility of the patient. Furthermore, current external braces have limited ability to correct the deformed spine and typically only prevent progression of the deformity. Thus, to avoid this need, doctors developed several internal fixation techniques to span across multiple vertebrae and force the spine into a desired orientation.

To fix the spine, surgeons attach one or more fixation elements (typically rods or plates) to the spine at several fixation sites, typically in the lumbar and sacral region, to correct and stabilize the spinal deformity, prevent reoccurrence of the spinal deformity, and stabilize weakness in trunks that results from degenerative discs and joint disease, deficient posterior elements, spinal fracture, and other debilitating problems. Where rods are used, they may be pre-curved or curved intraoperatively to a desired adjusted spinal curvature. Wires as well as bone screws or hooks can be used to pull individual vertebra or bone structure toward the rod, thereby anchoring the device to bone. The procedure may also include fusion of the instrumented spinal segments.

Once anchored, the rod-based systems are under stress and subjected to significant forces, known as cantilever pullout forces. As a result, surgeons are always concerned about the possibility of the implant loosening or the bone screws pulling out of the bone, especially where the system is anchored to the sacrum or ilium. The sacrum and ilium are usually of poor bone quality, consisting primarily of cancellous bone with thin cortical bone, magnifying the problem when fixation elements must be fixed to them. Thus, surgeons generally seek to attach implants in the most secure and stable fashion possible while at the same time addressing a patient's specific anatomy. While several current techniques exists for anchoring fixation elements to the sacrum and ilium, the current techniques require precise contouring and placement of spinal rods on the sacrum and/or ilium during surgery. The task becomes more difficult when, as is often called for, a surgeon must construct a framework of articulated spinal rods. As a result, while several different rod-based systems have been developed, they can be cumbersome, requiring complicated surgical procedures with long operating times to achieve correction. Furthermore, intraoperative adjustment of rod-based systems can be difficult and may result in loss of mechanical properties due to multiple bending operations. Surgeons find a number of the current techniques to be complex and challenging to implement.

Accordingly, there is a need in this art for novel implantable devices for correcting spinal deformities or degeneration that reduce the complexity of surgery, are compatible with current surgical techniques, and can be easily and intraoperatively customized.

### SUMMARY

The present invention generally provides various implantable devices and methods for correcting spinal deformities or degeneration. In one embodiment, a spinal connector is provided which includes a receiver head having opposed sidewalls defining a seating portion configured to seat a spinal fixation element. The seating portion can be in the form of, for example, opposed U-shaped slots formed between the opposed sidewalls of the receiver head. The spinal connector also includes a connecting rod extending from one of the sidewalls of the receiver head and having a first end pivotally coupled to the receiver head and a second end configured to couple to a spinal anchor.

In one exemplary embodiment, the first end of the connecting rod can be disposed within an opening formed in one of the sidewalls of the receiver head. An insert can be disposed within the receiver head and it can be pivotally coupled to the first end of the connecting rod. For example, the insert can include a post disposed through a bore formed in the first end of the connecting rod for allowing pivotal movement of the connecting rod about the post. In use, the insert can be adapted to lock the connecting rod in a fixed position relative to the receiver head when a spinal fixation element is locked within the receiver head. In one embodiment, at least a portion of the post of the insert and at least a portion of the bore can have a tapered shape adapted to allow an interference fit between the post and the bore when a spinal fixation element is locked within the receiver head. In another embodiment, at least a portion of the post of the insert can have a convex surface formed thereon and at least a portion of the bore of the receiver head can have a complementary concave surface formed thereon to allow an interference fit between the post and the bore when a spinal fixation element is locked within the receiver head.

In another embodiment of the invention, the spinal connector can have a receiver head with a closed configuration. For example, the spinal connector can have a rod seating portion in the form of a bore extending through the receiver head between the opposed sidewalls. The bore can be shaped to slidably receive a spinal fixation element therethrough. The spinal connector can also include a split ring disposed within the bore and configured to slidably receive the spinal fixation element therethrough.

Various spinal fixation systems are also provided, and in one exemplary embodiment the system can include a spinal connector having a receiver head with a seating portion, a connecting rod extending from the receiver head, and an elongate spinal fixation element having a portion mated to the seating portion of the spinal connector. In an exemplary embodiment, the elongate spinal fixation element can extend in a plane substantially parallel to a plane containing the connecting rod. At least one of the connecting rod and the elongate spinal fixation element can be pivotally coupled to the receiver head. The spinal connector can also include a split ring pivotally disposed within the receiver head for receiving the elongate spinal fixation element extending therethrough. In another embodiment, the spinal fixation system can include a spinal fixation plate having at least one thru-bore formed therethrough and adapted to receive a bone screw for anchoring the spinal fixation plate to bone. The spinal fixation plate can couple to a second end of the connecting rod of the spinal connector.

The spinal fixation system can also include an insert disposed within the receiver head and pivotally coupled to the connecting rod. The insert can include a post disposed through a bore formed in the first end of the connecting rod for allowing pivotal movement of the connecting rod about the post. In one embodiment, the insert can be adapted to lock the connecting rod in a fixed position relative to the receiver head when the elongate spinal fixation element is locked within the receiver head.

In yet another embodiment, the system can include a second spinal connector having a receiver head with a rod seating portion, and a connecting rod extending from the receiver head and having a first end pivotally coupled to the receiver head. The connecting rod of the second spinal connector can be disposed within the rod seating portion of the first spinal connector such that the connecting rod of the second spinal connector extends traverse to the connecting rod of the first spinal connector.

Exemplary methods for correcting spinal deformities are also provided, and in one embodiment the method can include coupling a connecting rod of a spinal connector to bone, for example, by mating the spinal connector to a spinal anchor implanted in bone, such as in iliac or sacral bone. A spinal fixation element can be positioned within a receiver head coupled to the connecting rod of the spinal connector. The method can further include pivoting at least one of the spinal fixation element and the connecting rod relative to the receiver head of the spinal connector, and locking the spinal fixation element within the receiver head thereby locking the receiver head and the connecting rod in a fixed position relative to one another. In one embodiment, the connecting rod can extend longitudinally along a spinal column such that it spans across a plurality of vertebrae, and the spinal rod can extend laterally. The connecting rod can be anchored to a plurality of vertebrae. The method can also include coupling a receiver head of a second spinal connector to the connecting rod of the first spinal connector, and anchoring a connecting rod pivotally coupled to the receiver head of the second spinal connector to bone. The connecting rod of the second spinal connector can be anchored to iliac or sacral bone.

### BRIEF DESCRIPTION OF THE DRAWINGS

The invention will be more fully understood from the following detailed description taken in conjunction with the accompanying drawings, in which:

FIG. 1 is an exploded view of one embodiment of a spinal connector having a receiver head and a connecting rod pivotally coupled to the receiver head;

FIG. 2 is an exploded view of another embodiment of a spinal connector having a receiver head and a connecting rod pivotally coupled to the receiver head;

FIG. 3 is an exploded view of yet another embodiment of a spinal connector having a receiver head and a connecting rod pivotally coupled to the receiver head;

FIG. 4 is an exploded view of a spinal connector having a receiver head coupled to a connecting rod, the receiver head including a bore formed therein and having an split ring for slidably and rotatably receiving a spinal fixation element in accordance with another embodiment;

FIG. 5 illustrates one exemplary embodiment of a spinal construct having longitudinal spinal fixation rods that are anchored to the ilium using first and second spinal connectors and first and second spinal fixation plates; and

FIG. 6 illustrates another exemplary embodiment of a spinal construct having first and second spinal connectors.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Various exemplary methods and devices are provided for connecting a spinal fixation construct to the spine, and preferably to the ilium and/or sacrum. In one embodiment of the invention, a spinal connector is provided having a receiver head with an opening for receiving a spinal fixation element, such as a spinal rod, and a connecting rod extending from the receiver head. The connecting rod can include a first end that is coupled to the receiver head, and a second end that is configured to couple to a spinal anchor, such as a plate or bone screw. In use, a spinal fixation element can be disposed within the receiver head of the spinal connector and the spinal connector can pivot relative to the spinal fixation element, or the connecting rod can pivot relative to the receiver head, to facilitate positioning and mating of the connecting rod to a spinal anchor. The spinal connector is particularly useful for anchoring a longitudinal spinal fixation element to sacral or iliac bone, as the pivotal movement between the spinal fixation element and the receiver head and/or the connecting rod and the receiver head allows the connecting rod to be easily disposed within and mated to an anchor implanted in sacral or iliac bone, thereby minimizing or eliminating the need to intra-operatively bend the spinal fixation element and/or connecting rod. A person having ordinary skill in the art will appreciate that, while the spinal connectors are particularly useful for anchoring a spinal construct to the sacrum or ilium, the spinal connectors and methods disclosed herein can be used in various portions of the spinal column for mating various implants.

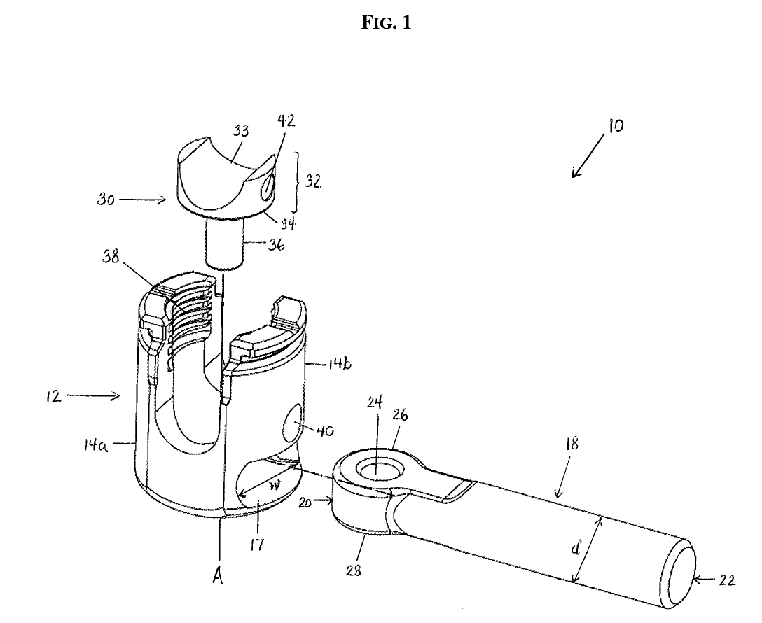

FIG. 1 illustrates one exemplary embodiment of a spinal connector. As shown, the spinal connector 10 generally includes a receiver head 12 having opposed sidewalls 14a, 14b defining a U-shaped recess 16 for seating a spinal fixation element. A connecting rod 18 extends from the receiver head 12 and includes a first end 20 pivotally coupled to the receiver head 12, and a second end 22 configured to couple to a spinal anchor. The receiver head 12 can also include an insert 30 disposed within the receiver head 12 for facilitating pivotal movement of the connecting rod 18, and optionally for locking a spinal fixation element within the receiver head 12 and/or locking the receiver head 12 and the connecting rod 18 in a fixed position relative to one another. As shown in FIG. 1, the insert 30 can include a post 36 that pivotally couples to the first end 20 of the connecting rod 18, and a rod seating portion 32 that is configured to seat a spinal fixation element disposed within the receiver head 12.

The receiver head 12 can have any size and shape to facilitate seating of a spinal fixation element, and the shape and size can vary depending on the type of spinal fixation element being used. In the illustrated embodiment, the receiver head 12 has a generally hollow U-shaped cylindrical configuration with an open proximal end and a closed distal end. Opposed slits are formed between opposed sidewalls 14a, 14b of the receiver head 12 to define a U-shaped recess 16 extending between the sidewalls 14a, 14b. The U-shaped recess 16 is configured to seat a generally elongate cylindrical spinal rod that is positioned between the opposed sidewalls 14a, 14b.

The receiver head 12 can also include an opening 17 formed therein for receiving the connecting rod 18. As shown in FIG. 1, the opening 17 is formed in the sidewall 14b adjacent to the distal end of the receiver head 12. As a result, the connecting rod 18, which will be discussed in more detail below, will extend outward from the receiver head 12 in a direction that is substantially perpendicular to a spinal fixation element seated in and extending through the receiver head 12. The connecting rod 18 will also be positioned in a plane that is spaced distal of and distance apart from a plane containing a spinal fixation element seated in and extending through the receiver head 12. A person skilled in the art will appreciate that the opening 17 can be formed at various other locations. For example, the connecting rod 18 can extend parallel to or at other angles relative to a spinal fixation element positioned within the receiver head 12.

The connecting rod 18 can also have a variety of configurations, but as shown in FIG. 1, it has a generally elongate cylindrical configuration with a first end 20 that is disposable within the opening 17 in the receiver head 12, and a second end 22 that is adapted to mate to a spinal anchor, such as a hook, a plate, a bone screw, or other devices for anchoring the second end 22 to bone. In an exemplary embodiment, the first end 20 is pivotally coupled to the receiver head 12 to allow the connecting rod 18 and the receiver head 12 to be angularly adjusted relative to one another. While various techniques can be used to allow pivotal movement, in the illustrated embodiment the first end 20 has a generally circular or cylindrical shape that is sized to fit within a circular or cylindrical cavity formed within the receiver head 12. In an exemplary embodiment, an outer diameter of the first end 20 is only slightly less than an inner diameter of the receiver head 12 such that a clearance fit is formed between the two components. The first end 20 can also have an the inferior surface 28 that can rest against an interior surface of the closed distal end of the receiver head 12, and a superior surface 26 that seats the insert 30, which will be discussed in more detail below. While the shape of the surfaces 26, 28 can vary, in the illustrated embodiment the superior and inferior surfaces 26, 28 are substantially planar. The first end 20 can also include a bore 24 formed therethrough and extending between the superior and inferior surfaces 26, 28 thereof. The bore 24 is configured to receive a post 36 of the insert 30, which will be discussed in more detail below. The remainder of the connecting rod 18 that extends from the first end 20 can have various shapes and sizes, but in one exemplary embodiment, as shown, the connecting rod 18 is in the form of an elongate cylindrical rod. The length of the connecting rod 18 can vary depending on the intended use. The diameter d of the connecting rod 18 can also vary, but preferably, the diameter d of the connecting rod 18 is less than the width w of the opening 17 of the receiver head 12 to allow the connecting rod 18 to pivot when it is disposed in the receiver head 12. In an exemplary embodiment, the difference between the diameter d and the width w is sufficient to allow the connecting rod 18 to pivot over a range of about 50 degrees. A person skilled in the art will appreciate that the diameter d of the connecting rod 18 and the width w of the opening 17 in the receiver head 12 can have any dimension that provides for pivotal movement between the connecting rod 18 and the receiver head 12, as may be desired.

In order to mate the connecting rod 18 to the receiver head 12, the receiver head 12 can also include an insert 30 disposed therein. As shown in FIG. 1, the insert 30 has a generally cylindrical shape that is sized to fit within the receiver head 12. In an exemplary embodiment, the insert 30 can include a rod seating portion 32 configured to be aligned with the U-shaped recess 16 and to seat a spinal fixation element. For example, a superior surface 33 of the rod seating portion 32 of the insert 30 can include a concave recess for seating a spinal fixation element, or it can have other shapes to match a contour of a spinal fixation element. The insert 30 can also include an inferior surface 34 with a post 36 extending therefrom. The post 36 is configured to be disposed through the bore 24 in the connecting rod 18 to allow for pivotal movement of the connecting rod 18 about the post 36. When mated, the inferior surface 34 of the rod seating portion 32 can sit against the superior surface 26 of the connecting rod 18 when the post 36 is inserted into the bore 24. The insert 30 can also be adapted to lock the connecting rod 18 and the receiver head 12 in a fixed position relative to one another, as will be discussed in more detail below.

The receiver head 12 and the insert 30 can also optionally include features for retaining the insert 30 in the receiver head 12 and thereby preventing the connecting rod 18 from being removed from or falling out of the receiver head 12. For example, the receiver head 12 can include opposed bores (only one bore 40 is shown) having a deformable material (not shown) disposed therein and extending there across. The bores allow the material to be deformed inward to extend into and engage corresponding detents (only one detent 42 is shown) formed in the insert 30. A tool can be used to deform the material into the detents once the insert 30 is disposed within the receiver head 12. As a result, the insert 30 can be maintained within the receiver head 12, thereby preventing removal of the insert 30 and thus the connecting rod 18 from the receiver head 12. A person skilled in the art will appreciate that a variety of techniques can be used to retain the insert 30 within the receiver head 12, such as retaining the insert 30 within the receiver head 12 using a cross-pin. Moreover, any number of bores and corresponding detents can be used to retain the insert 30 in the receiver head 12.

When the device is assembled, the connecting rod 18 extends through the opening 17 in the receiver head 12 such that the first end 20 sits within the receiver head 12. The post 36 of the insert 30 extends through the bore 24 of the connecting rod 18, with the inferior surface 34 of the rod seating portion 32 resting against the superior surface 26 of the bore 24. The connecting rod 18 and the receiver head 12 can pivot relative to one another and about an axis A extending through the receiver head 12 and the bore 24, thereby facilitating mating of the connecting rod 18 to a spinal anchor after a spinal fixation element, such as a spinal rod, is positioned within the receiver head 12, or alternatively facilitating positioning of a spinal fixation element within the receiver head 12 after the connecting rod 18 is anchored to bone. In particular, the connecting rod 18 can be pivoted relative to the receiver head 12 when a spinal fixation element is mated to the receiver head 12, or the receiver head 12 can be pivoted relative to the connecting rod 18 when the second end 22 of the connecting rod is coupled to a spinal anchor.

As previously indicated, the insert 30 can be adapted to lock the connecting rod 18 and the receiver head 12 in a fixed position relative to one another. For example, the spinal fixation element can be effective to lock the insert 30 and the connecting rod 18 by bearing against the insert 30, which in turn bears against the connecting rod 18 causing the connecting rod 18 to remain in a fixed position with respect to the receiver head 12. In particular, once a spinal rod or other spinal fixation element is positioned within the receiver head 12, a locking mechanism can optionally be applied to the receiver head 12 to lock the spinal fixation element therein. While various locking techniques can be used, in the embodiment shown in FIG. 1, the receiver head 12 can include threads formed on an interior surface of a proximal portion of each sidewall for mating with corresponding threads on a locking mechanism. For example, FIG. 1 illustrates threads 38 formed on the interior surface of sidewalls 14a, 14b adjacent to the open proximal end of the receiver head 12. The locking mechanism (not shown) can be, for example, a threaded nut or set screw that can threadably mate to the sidewalls 14a, 14b to apply a downward pressure on a spinal rod disposed within the receiver head 12, thereby locking the spinal rod within the U-shaped recess 16 of the receiver head 12. As a result, the spinal rod is prevented from sliding and rotating relative to the spinal connector 10. A person skilled in the art will appreciate that various locking mechanisms, such as snap-lock and twist-lock mechanisms, are known in the art and can be used with the spinal connectors disclosed herein. Moreover, the locking mechanism can mate to an external surface of each sidewall, or to both external and internal portions of each sidewall.

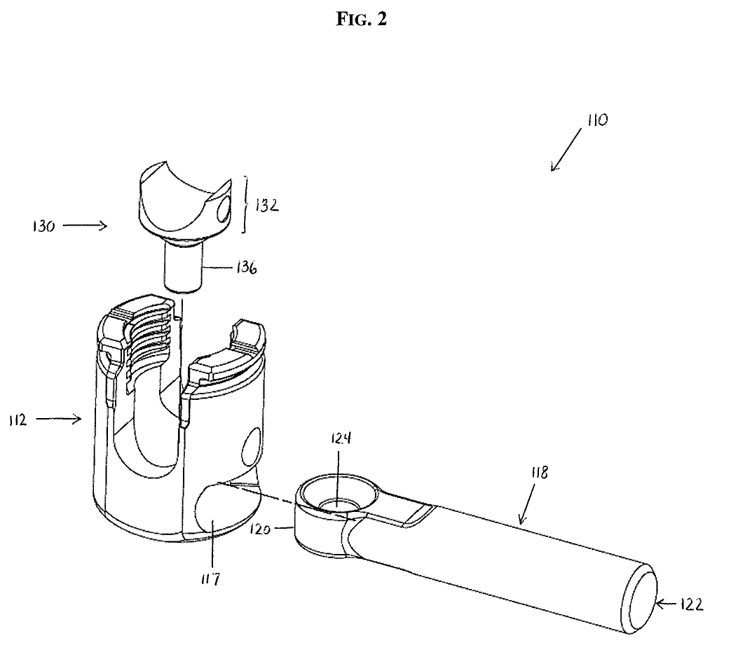

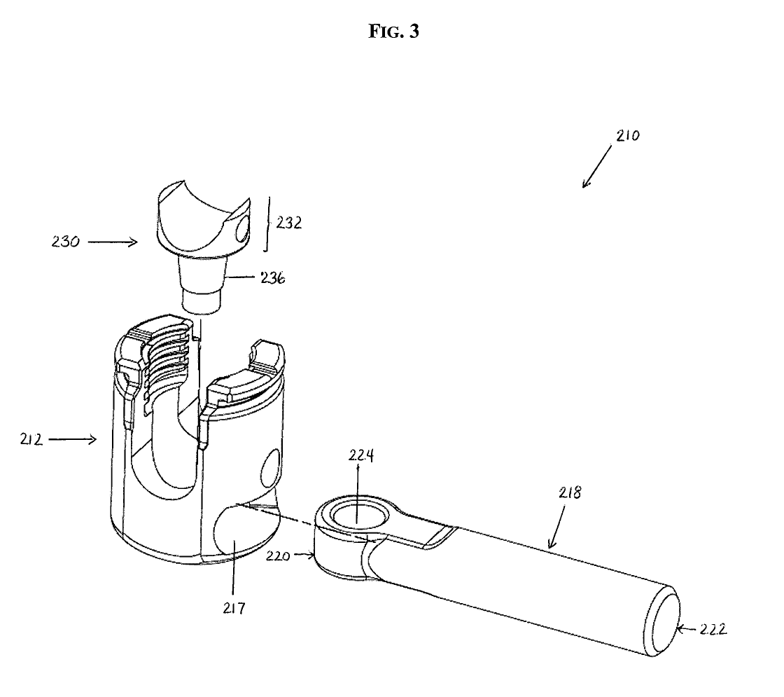

In other embodiments, the post 36 and the bore 24 can be shaped to further facilitate locking of the connecting rod and the receiver head. By way of non-limiting example, FIGS. 2 and 3 each illustrate one exemplary embodiment of a spinal connector having a post and a bore shaped to facilitate locking. As shown in FIGS. 2-3, the spinal connectors 110, 210 are similar to the spinal connector 10 shown in FIG. 1, and they generally include a receiver head 112, 212 having an insert 130, 230, and a connecting rod 118, 218 with a first end 120, 220 disposed in an opening 117, 217 formed in the receiver head 112, 212, and a second end 122, 222 configured for coupling to a spinal anchor. In the embodiment shown in FIG. 2, at least a portion of a bore 124 formed in the first end 120 of the connecting rod 118 has a spherical shape for receiving a post 136 of the insert 130. The inferior surface of a rod seating portion 132 of the insert 130 has a spherical shape that complements the spherical shape of the bore 124, allowing for an interference fit when a spinal fixation element is locked within the receiver head 112. In the embodiment shown in FIG. 3, at least a portion of a post 236 of an insert 230 and at least a portion of a bore 224 of the connecting rod 218 each have a tapered configuration, also allowing for an interference fit when a spinal fixation element is locked within the receiver head 212. A person skilled in the art will appreciate that the post of the insert and the bore of the connecting rod can have a variety of configurations adapted to facilitate locking of the connecting rod and the receiver head, including threaded or stepped configurations.

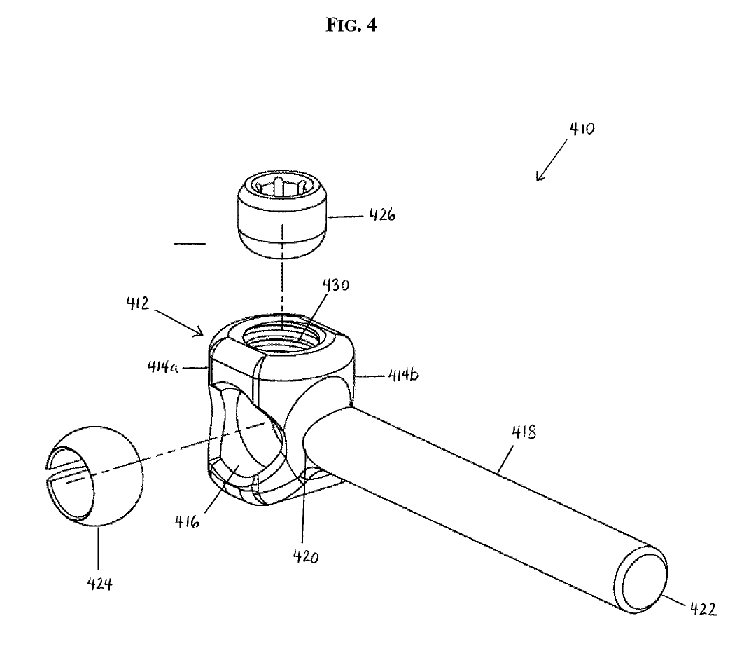

In another embodiment, rather than providing a connecting rod and receiver head that pivot relative to one another, the spinal connector can be configured to allow a spinal fixation element to pivot relative to the receiver head to thereby facilitate mating of the second end of the connecting rod to a spinal anchor. By way of non-limiting example, FIG. 4 illustrates one such spinal connector. As shown in FIG. 4, the spinal connector 410 includes a receiver head 412 having a connecting rod 418 with a first end 420 that is integrally formed with or coupled to the receiver head 412, and a second end 422 configured to couple to a spinal anchor. A person skilled in the art will appreciate that the connecting rod 418 can couple to the receiver head 412 using a variety of techniques, including both fixed and adjustable mating techniques. The receiver head 412 can also include a rod seating portion in the form of a bore 416 extending therethrough between opposed sidewalls 414a, 414b of the receiver head 412. The bore 416 can have a central axis that extends in a direction substantially perpendicular to an axis of the connecting rod 418 mated to the receiver head 412, although a person skilled in the art will appreciate that the orientation of the bore 416 with respect to the connecting rod 418 can vary. The bore 416 is sized and shaped to slidably receive a spinal fixation element therethrough, and to allow the spinal fixation element to pivot (i.e., move polyaxially) relative to the receiver head 412. The bore 416 can also include a split ring, such as a spherical-shaped split ball ring 424 disposed therein for receiving the spinal fixation element. The split ring 424 can be sized and shaped to allow it to pivot within the bore 416, which in effect allows the spinal fixation element disposed within the split ring 424 to pivot relative to the receiver head 412. In the exemplary embodiment, the split ring 424 has a generally spherical exterior surface to allow it to pivot within the bore 416 of the receiver head 412. A person skilled in the art will appreciate that the split ring 424 can have a variety of configurations to facilitate its pivotal movement within the bore 416.

Once a spinal rod or other spinal fixation element is positioned within split ring 424 of the bore 416 formed in the receiver head 412, the split ring 424 can pivot to position the spinal fixation element relative to the receiver head 412 to facilitate coupling of the second end 422 of the connecting rod 418 to a spinal anchor. To fix the position of the spinal fixation element and the receiver head 412 relative to one another, a locking mechanism can be applied to the receiver head 412 to lock the spinal fixation element therein. While various locking techniques can be used, in the embodiment shown in FIG. 4 the receiver head 412 includes threads 430 formed on an interior surface of a proximal portion of the receiver head 412 for mating with corresponding threads on a locking mechanism 426. The locking mechanism 426 can be, for example, a threaded nut or set screw 426, as shown, that can threadably mate to the side walls 414a, 414b. This will apply a downward pressure on the split ring 424 within the bore 416 to compress the split ring 424 causing it to engage a spinal rod disposed therein and thereby lock the spinal rod within the bore 416 of the receiver head 412. As a result, the split ring and the spinal rod are prevented from sliding and rotating relative to the receiver head 412. A person skilled in the art will appreciate that various locking mechanisms, such as snap-lock and twist-lock mechanisms, are known in the art and can be used with the spinal connectors disclosed herein. Moreover, the locking mechanism can mate to internal and/or external surfaces of the receiver head 412.

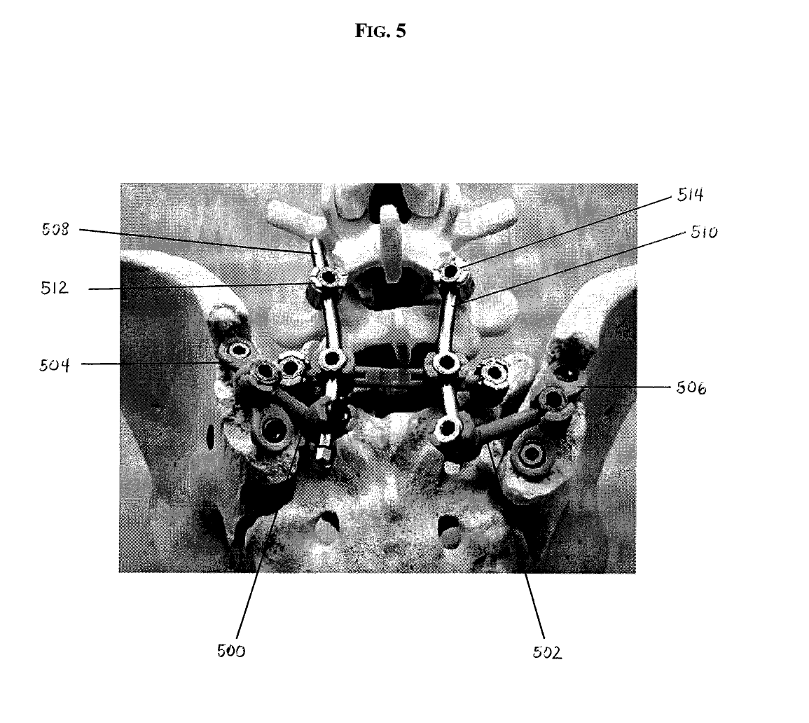

FIG. 5 illustrates one exemplary use of a spinal connector shown mated to various implants to form a spinal fixation construct. While the methods and constructs are described in connection with the spinal connector 10 shown in FIG. 1, a person skilled in the art will appreciate that the spinal connector can have virtually any configuration, and that the particular configuration can vary depending on the intended use. Moreover, the components used in each construct and the particular configuration of each component can vary. Various other devices known in the art can also be used to provide certain mating connections between the components of the various constructs.

In the embodiment shown in FIG. 5, first and second spinal connectors 500, 502 are used to couple first and second longitudinal spinal rods 508, 510 to first and second spinal anchors 504, 506 implanted in the ilium. In particular, first and second spinal rods 508, 510 are shown extending longitudinally along the spine such that they span across multiple adjacent vertebrae. Each rod 508, 510 can be anchored to one or more vertebrae. FIG. 5 illustrates a first bone screw 512 for anchoring the first rod 508 to a first lateral side of a vertebra in the cervical spine, and a second bone screw 514 for anchoring the second rod 510 to the opposed lateral side of the vertebra. In order to anchor the rods 508, 510 to the ilium, a terminal end of each rod 508, 510 can be positioned within a receiver head of a spinal connector 500, 502, as shown. Each spinal connector 500, 502 can pivot relative to the rods 508, 510, or each connecting rod of the spinal connectors 500, 502 can pivot relative to each receiver head to facilitate positioning and mating of the connecting rods to spinal anchors 504, 506 implanted in the ilium. This allows the connecting rods to mate to the spinal anchors 504, 506 without having to bend or deform the longitudinal spinal rods 508, 510 or the connecting rods of the spinal connectors 500, 502. The anchors are described in more detail in a U.S. Patent Application filed on even date herewith and entitled “Articulating Sacral or Iliac Connector,” by Nam T. Chao, Peter Newton, Randal Betz, and Tim Mondeau (Attorney Docket No. 101896-469), which is hereby incorporated by reference in its entirety. In general, each spinal anchor 504, 506 includes a plate having an elongate configuration with opposed thru-bores formed therein that are configured to receive a bone screw for attaching to a bone, such as the iluim, as shown in FIG. 5. Each spinal anchor also includes a receiving portion formed thereon or removably mated thereto for mating to the spinal connectors 500, 502. The connecting rod of each spinal connector 500, 502 can couple to the receiving portion of each spinal anchors 504, 506 to anchor the spinal connectors 500, 502 to bone. A person skilled in the art will appreciate that a variety of spinal anchors can be used in the spinal fixation construct, including hooks, bone screws, and plates. To lock the rods 508, 510 within the receiver head of each spinal connector 500, 502, and to lock the position of the connecting rods relative to the receiver heads of each spinal connector 500, 502, a locking mechanism, such as a set screw, can be threadably mated to the receiver head of each spinal connector 500, 502. This will apply a downward pressure on the rods 508, 510 causing the receiver heads and the rods 508, 510 disposed therein to engage one another, thereby locking the rods 508, 510 within the receiver head. This downward pressure also acts to lock the connecting rods and the receiver heads relative to one another.

The spinal construct can also optionally include a spinal cross-connector, which is described in more detail in a U.S. Patent Application filed on even date herewith and entitled “Sliding Sacral or Iliac Connector,” by Nam T. Chao, Munish Gupta, and Ross Sylvia (Attorney Docket No. 101896-471), which is hereby incorporated by reference in its entirety. In general, the cross-connector 512 includes first and second receiver heads slidably disposed along a spinal fixation element or rod. Each receiver head is effective to mate to the longitudinal rods 508, 510.

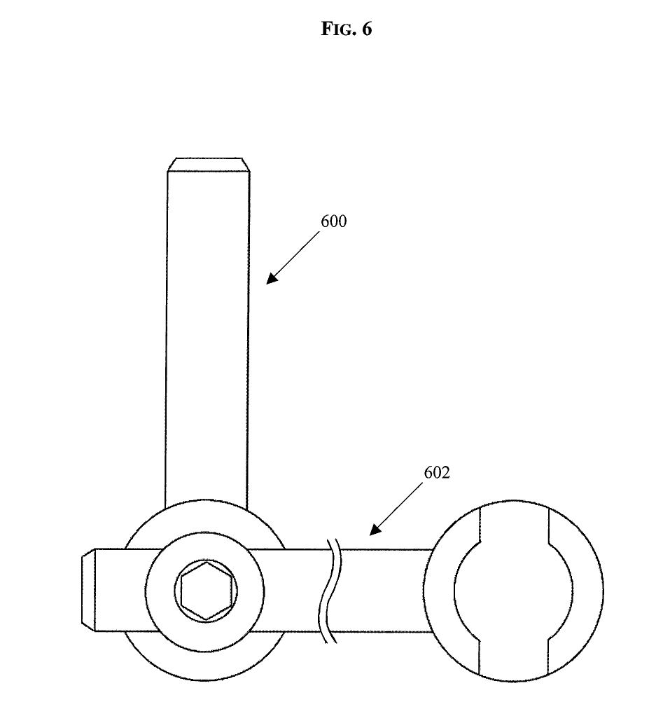

A person skilled in the art will appreciate that the spinal connectors described herein can be used in a variety of different spinal constructs. For example, as shown in FIG. 6, a spinal construct can include first and second spinal connectors 600, 602 as described above, both having a receiver head with a rod seating portion, and a connecting rod extending from the receiver head. The first spinal connector 600 can extend laterally with respect to the spine, and the second spinal connector 602 can extend longitudinally along the spine. The connecting rod of the second spinal connector 602 can be disposed within and mated to the rod seating portion of the first spinal connector 600. Additional components, including spinal fixation devices and spinal anchors, can be coupled to the first and second spinal connectors to couple the spinal construct to one or more vertebrae and/or to the iluim or sacrum.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A spinal connector, comprising:
a receiver head having opposed sidewalls defining a seating portion configured to seat a spinal fixation element; and
a connecting rod extending from one of the sidewalls of the receiver head and having a first end pivotally coupled to the receiver head and a second end configured to couple to a spinal anchor.

2. The spinal connector of claim 1, wherein the first end is disposed within an opening formed in one of the sidewalls of the receiver head.

3. The spinal connector of claim 1, further comprising an insert disposed within the receiver head and pivotally coupled to the first end of the connecting rod.

4. The spinal connector of claim 3, wherein the insert has a post disposed through a bore formed in the first end of the connecting rod for allowing pivotal movement of the connecting rod about the post.

5. The spinal connector of claim 4, wherein the insert is adapted to lock the connecting rod in a fixed position relative to the receiver head when a spinal fixation element is locked within the receiver head.

6. The spinal connector of claim 5, wherein at least a portion of the post of the insert and at least a portion of the bore have a tapered shape adapted to allow an interference fit between the post and the bore when a spinal fixation element is locked within the receiver head.

7. The spinal connector of claim 5, wherein at least a portion of the post of the insert has a convex surface formed thereon and at least a portion of the bore of the receiver head has a complementary concave surface formed thereon to allow an interference fit between the post and the bore when a spinal fixation element is locked within the receiver head.

8. The spinal connector of claim 1, wherein the rod seating portion comprises opposed U-shaped slots formed between the opposed sidewalls of the receiver head.

9. The spinal connector of claim 1, wherein the rod seating portion comprises a bore extending through the receiver head between the opposed sidewalls, the bore being shaped to slidably receive a spinal fixation element therethrough.

10. The spinal connector of claim 9, further comprising a split ring disposed within the bore and configured to slidably receive the spinal fixation element therethrough.

11. A spinal fixation system, comprising:
a spinal connector comprising a receiver head having a seating portion, and a connecting rod extending from the receiver head; and
an elongate spinal fixation element having a portion mated to the seating portion of the spinal connector, the elongate spinal fixation element extending in a plane substantially parallel to a plane containing the connecting rod;
wherein at least one of the connecting rod and the elongate spinal fixation element are pivotally coupled to the receiver head.

12. The spinal fixation system of claim 11, wherein the connecting rod has a first end that is pivotally coupled to the receiver head.

13. The spinal fixation system of claim 11, further comprising a split ring pivotally disposed within the receiver head, the elongate spinal fixation element extending through the split ring.

14. The spinal fixation system of claim 11, further comprising a spinal fixation plate having at least one thru-bore formed therethrough and adapted to receive a bone screw for anchoring the spinal fixation plate to bone, the spinal fixation plate coupled to a second end of the connecting rod of the spinal connector.

15. The spinal fixation system of claim 11, further comprising an insert disposed within the receiver head and pivotally coupled to the connecting rod.

16. The spinal fixation system of claim 15, wherein the insert has a post disposed through a bore formed in the first end of the connecting rod for allowing pivotal movement of the connecting rod about the post.

17. The spinal fixation system of claim 15, wherein the insert is adapted to lock the connecting rod in a fixed position relative to the receiver head when the elongate spinal fixation element is locked within the receiver head.

18. The spinal fixation system of claim 11, wherein the spinal connector comprises a first spinal connector, and the system further comprises a second spinal connector having a receiver head with a rod seating portion, and a connecting rod extending from the receiver head and having a first end pivotally coupled to the receiver head, the connecting rod of the second spinal connector being disposed within the rod seating portion of the first spinal connector such that the connecting rod of the second spinal connector extends traverse to the connecting rod of the first spinal connector.

19. A method for correcting spinal deformities, comprising:
coupling a connecting rod of a spinal connector to bone;
positioning a spinal fixation element within a receiver head coupled to the connecting rod of the spinal connector;
pivoting at least one of the spinal fixation element and the connecting rod relative to the receiver head of the spinal connector; and
locking the spinal fixation element within the receiver head thereby locking the receiver head in a fixed position relative to the connecting rod.

20. The method of claim 19, wherein coupling the spinal connector to bone comprises mating the spinal connector to a spinal anchor implanted in bone.

21. The method of claim 20, wherein the spinal anchor is implanted in iliac or sacral bone.

22. The method of claim 19, wherein the connecting rod extends longitudinally along a spinal column such that it spans across a plurality of vertebrae, and wherein the spinal rod extends laterally.

23. The method of claim 22, further comprising anchoring the connecting rod to a plurality of vertebrae.

24. The method of claim 22, wherein the spinal connector comprises a first spinal connector, and the method further comprises coupling a receiver head of a second spinal connector to the connecting rod of the first spinal connector, and anchoring a connecting rod pivotally coupled to the receiver head of the second spinal connector to bone.

25. The method of claim 24, wherein the connecting rod of the second spinal connector is anchored to iliac or sacral bone.
